(19) United States
(12) Patent Application Publication
(10) Pub. No.: US 2022/0331026 A1
(43) Pub. Date: Oct. 20, 2022

(54) ROBOTIC SURGICAL INTERVENTION DEVICE WITH CONTROLLED ARTICULATED ARM FOR FOLLOWING A PATH

(71) Applicant: COLLIN, BAGNEUX (FR)

(72) Inventors: Stéphane MAZALAIGUE, BAGNEUX (FR); Armand CZAPLINSKI, BAGNEUX (FR)

(21) Appl. No.: 17/764,375

(22) PCT Filed: Oct. 1, 2020
(86) PCT No.: PCT/FR2020/051724

§ 371 Date: May 2, 2022

(30) Foreign Application Priority Data
Oct. 4, 2019 (FR) FR1911028

(51) Int. Cl. A61B 34/30; A61B 34/00

(52) U.S. Cl. CPC A61B 34/30; A61B 34/70

(57) ABSTRACT

A robotic surgical intervention device includes an articulated arm, with actuating motors, a distal end of which is intended to carry a surgical instrument, a control peripheral of the articulated arm for moving a functional end of the surgical instrument along a path, and a processor for processing movement instructions provided by the control peripheral to convert them into instructions for controlling the actuating motors of the articulated arm. It further includes a trigger for automatic reversal of the articulated arm, the actuation of which causes individual reverse control instructions to be sent to each of the actuating motors of the articulated arm for a reverse movement of the functional end of the surgical instrument along the completed path.

The present invention relates to a robotic device for surgical intervention, particularly in the field of otorhinolaryngology but not only.

It applies more specifically to a robotic device comprising:
an articulated arm with actuating motors, a distal end of which is intended to carry a surgical instrument;
an articulated arm control peripheral for moving a functional end of the surgical instrument along a path; and
means for processing movement instructions provided by the control peripheral to convert them into individual control instructions for controlling each of the actuating motors of the articulated arm.

Such a device is described in the paper by Miroir et al, entitled “RobOtol: from design to evaluation of a robot for middle ear surgery”, published at the IEEE/RSJ International Conference on Intelligent Robots and Systems held on Oct. 18-22, 2010 in Taipei, Taiwan. It presents an architecture and kinematics particularly well suited to otological surgical interventions on the middle or inner ear of patients. These interventions are sensitive to wrong movements, so that robotic assistance is a valuable aid.

Nevertheless, even with this assistance, the practitioner may make a wrong gesture when manipulating the control peripheral, given the confined volume in which he or she must generally operate. If in most cases, the precision of the surgical gesture is such that a slight deviation is of no consequence and can easily be corrected, there are particular situations in which the tolerance is zero or almost zero. This is the case, for example, when disengaging an otologic surgical instrument inside a patient's ear. The path followed by the functional end of the surgical instrument for its engagement can be complex, given the crevices in which it is brought to evolve, so that its removal can be tricky if it must take into account the completed path. Moreover, such a removal can be desired in an emergency situation so that it must be fast. This speed of execution adds stress for the practitioner and an increased risk of wrong gesture.

More generally, in any type of surgical intervention assisted by a robotic device carrying a surgical instrument and manipulated with the help of a control peripheral, situations in which the slightest inaccuracy in case of a desired rapid removal of a surgical instrument can have serious consequences are numerous.

It may thus be desired to provide a robotic device that makes it possible to avoid at least some of the abovementioned problems and constraints.

A robotic surgical intervention device is therefore proposed, comprising:
an articulated arm with actuating motors, a distal end of which is intended to carry a surgical instrument;
a control peripheral for controlling the articulated arm for moving a functional end of the surgical instrument along a path; and
means for processing movement instructions provided by the control peripheral to convert them into individual control instructions for controlling each of the actuating motors for actuating the articulated arm;
   further comprising a trigger for automatic reversal of the articulated arm, the actuation of which causes, independently of any movement instruction of the control peripheral, the sending of individual reverse control instructions to each of the actuating motors of the articulated arm for a reverse movement of the functional end of the surgical instrument along the completed path.

In this way, any reversal can be carried out automatically by trigger and without the help of the control peripheral, with the guarantee that the reverse path is strictly followed. In the tricky situations mentioned above, this prevents any deviation from the desired retractions, even in restricted volumes or with crevices and regardless of any instructions issued by the control peripheral when the automatic reversal is triggered.

Optionally, the robotic device is configured so that actuation of the automatic reversal trigger causes a suspension of processing of new movement instructions provided by the control peripheral.

Also optionally:
the processing means include means for storing in memory an ordered series of successive positionings of the articulated arm during controlled movement of the functional end along the path; and
the processing means are configured so that the actuation of the automatic reversal trigger causes successive reversals of the articulated arm step by step from a last stored positioning to a first stored positioning of this ordered series.

Also optionally, each stored positioning of the ordered series of successive positionings of the articulated arm includes a set of positions of its actuating motors.

Also optionally, the storing means are configured to store in memory the successive positionings of the articulated arm at regular time intervals.

Also optionally, the memory in which the ordered series of successive positionings of the articulated arm is stored is configured in a stack structure.

Also optionally, the automatic reversal trigger includes a stop pedal or push button with a speed variator for varying the speed of the automatic reversal of the articulated arm as a function of pressure exerted by an operator.

Also optionally, the processing means are configured to stop the current automatic reversal and resume processing new movement instructions provided by the control peripheral as soon as no pressure is any more applied to the automatic reversal trigger by the operator.

Also optionally, the control peripheral is a 6D joystick.

Also optionally, a robotic surgical intervention device according to the invention may be configured and sized for a middle or inner ear surgical intervention on a patient, the surgical instrument itself being a patient's middle or inner ear surgical intervention instrument.

The invention will be better understood with the aid of the following description, which is given only by way of example and is made with reference to the appended drawings wherein:

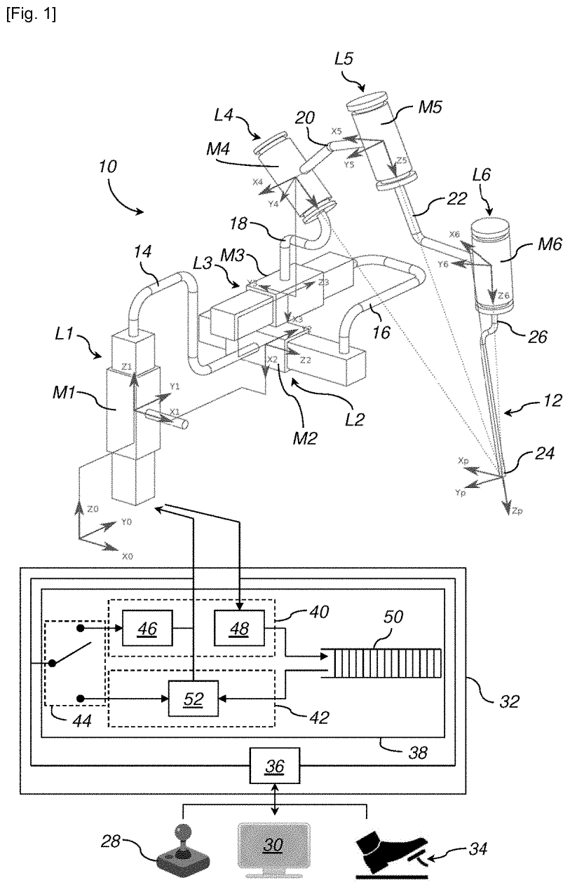

FIG. 1 diagrammatically represents the general structure of a robotic surgical intervention device, according to an embodiment of the invention,

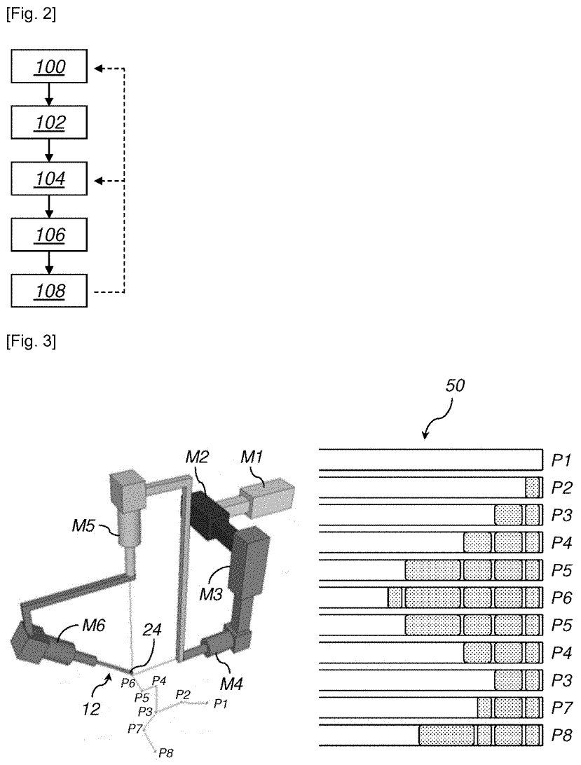

FIG. 2 illustrates the successive steps of a surgical intervention method using the robotic device of FIG. 1, according to an embodiment of the invention, and

FIG. 3 illustrates an example of a scenario realized by executing the method of FIG. 2.

With reference to FIG. 1, a robotic surgical intervention device according to an embodiment of the invention comprises an articulated arm 10 with actuating motors carrying a surgical instrument 12. The non-limiting example illustrated in this figure is more specifically that of a robotic device for an application in otological surgery of the middle or inner ear of a patient, the architecture and kinematics of which are optimized in accordance with the teaching of the aforementioned document by Miroir et al. The articulated arm 10 thus has, from its base to its end carrying the surgical instrument 12, three motorized prismatic links in series followed by three motorized rotoid links in series.

A first prismatic link L1, actuated by a first motor M1, allows translational movement of a first member 14 of the articulated arm 10 along the axis Z1 (for example vertical) of a first local orthogonal Cartesian coordinate system (X1, Y1, Z1) linked to the first motor M1. The first motor M1 is attached to the robotic device so that the first local coordinate system (X1, Y1, Z1) has the same directions as a global orthogonal Cartesian coordinate system (X0, Y0, Z0) linked to a fixed base of the robotic device. The movement axis of the first member 14 is thus parallel to Z0.

A second prismatic link L2, actuated by a second motor M2 carried by one end of the first member 14, allows translational movement of a second member 16 of the articulated arm 10, along the axis Z2 of a second local orthogonal Cartesian coordinate system (X2, Y2, Z2) linked to the second motor M2. The second local coordinate system (X2, Y2, Z2) is turned through a right angle with respect to the Y1 axis of the first local coordinate system (X1, Y1, Z1) so that its Z2 axis is parallel to the X1 axis. The movement axis of the second member 16 is thus parallel to X0.

A third prismatic link L3, actuated by a third motor M3 carried by one end of the second member 16, allows translational movement of a third member 18 of the articulated arm 10, along the axis Z3 of a third local orthogonal Cartesian coordinate system (X3, Y3, Z3) linked to the third motor M3. The third local coordinate system (X3, Y3, Z3) is turned through a right angle with respect to the X2 axis of the second local coordinate system (X2, Y2, Z2) so that its Z3 axis is parallel to the Y2 axis which is itself parallel to the Y1 axis. The movement axis of the third member 18 is thus parallel to Y0.

A fourth rotoid link L4, actuated by a fourth cylindrical motor M4 and carried by one end of the third member 18, allows the rotational movement of a fourth member 20 of the articulated arm 10, about the axis Z4 of a fourth local orthogonal Cartesian coordinate system (X4, Y4, Z4) linked to the fourth motor M4.

A fifth rotoid link L5, actuated by a fifth cylindrical motor M5 and carried by one end of the fourth member 20, allows the rotational movement of a fifth member 22 of the articulated arm 10, about the axis Z5 of a fifth local orthogonal Cartesian coordinate system (X5, Y5, Z5) linked to the fifth motor M5.

Finally, a sixth rotoid link L6, actuated by a sixth cylindrical motor M6 and carried by one end of the fifth member 22, allows the rotational movement of the surgical instrument 12, about the axis Z6 of a sixth local orthogonal Cartesian coordinate system (X6, Y6, Z6) linked to the sixth motor M6.

According to the particularly interesting configuration of FIG. 1, the three respective rotation axes Z4, Z5 and Z6 of the three rotoid links converge at a same central point of the functional distal end 24 of the surgical instrument 12, thus making this point a pivot point. This means that in the absence of any actuation of the motors M1, M2, M3 of the prismatic links, any instruction to actuate at least one of the motors M4, M5, M6 of the rotoid links causes a rotation of the surgical instrument 12 about its pivot point without any movement of the latter in the global coordinate system (X0, Y0, Z0).

The surgical instrument 12 has a proximal end 26 for attachment to the articulated arm 10, more specifically to a corresponding attachment end of the arm 10 linked to the motor M6. This attachment is, for example, advantageously made in accordance with the locking system described in patent FR 2 998 344 B1, but this is not mandatory. Any other fastening system suitable for the intended application is also suitable.

The surgical instrument 12 may have a rectilinear shape such that its main axis Zp, about which a local Cartesian coordinate system (Xp, Yp, Zp) is defined and linked to it, is that which connects a central point of its proximal attachment end 26 to the pivot point of its distal functional end 24. In this case, not shown in FIG. 1, the Zp axis merges with the Z6 axis.

Alternatively, and as illustrated in FIG. 1, it may be a surgical instrument with deviated portions such as that described in patent application FR 3 066 378 A1. In this case, its main axis Zp, around which the local Cartesian coordinate system (Xp, Yp, Zp) linked to it is still defined, is that of a rectilinear distal portion of this instrument, off-axis with respect to the axis Z6 which still connects the central point of its proximal fixing end 26 to the pivot point of its functional distal end 24.

The robotic surgical intervention device further comprises a peripheral control device 28 for the articulated arm 10, such as a 6D joystick or any other equivalent device, adapted to allow a movement of the functional distal end 24 of the surgical instrument 12 along a desired path according to three translational degrees of freedom and three rotational degrees of freedom by actuating the six motors M1 to M6. It may also include a screen 30, in particular for displaying and monitoring any movement of the surgical instrument 12 during the operating phase along its path.

The robotic surgical intervention device further comprises means for processing movement instructions provided by the control peripheral 28 in order to convert them into individual control instructions for controlling each of the motors M1 to M6 of the articulated arm 10. These processing means take the form of an electronic circuit 32.

The robotic surgical intervention device further comprises a trigger 34 for automatic reversal of the articulated arm 10. For example, it can be a stop pedal or push-button device with speed variator for varying the speed of the automatic reversal of the articulated arm 10 as a function of a pressure exerted by an operator. Its function when actuated, for example by foot pressure if it is a pedal, is to cause, independently of any movement instruction from the control peripheral 28, the sending of individual reverse control instructions to each of the six motors M1 to M6 for a reverse movement of the functional end 24 of the surgical instrument 12 along the completed path. In surgical practice, the trigger 34 is advantageously a pedal since its actuation by the foot frees the practitioners hands.

The electronic circuit 32 is connected to the articulated arm 10 in order to transmit thereto the individual control instructions for controlling the motors M1 to M6 and to receive back as often as it wishes the cartesian or angular positions of the motors M1 to M6. It is connected to the control peripheral 28 in order to receive its movement instructions. These are generally expressed in the global coordinate system (X0, Y0, Z0). It is connected to the reversal trigger 34 to detect its actuation and consequently to engage the automatic reversal of the articulated arm 10.

It has a central processing unit 36, such as a microprocessor designed to transmit to the articulated arm 10 the individual control instructions, to receive from the control peripheral 28 the movement instructions and to receive from the articulated arm 10 the positions of the motors M1 to M6. It further has a memory 38 wherein at least one computer program is stored, to be executed by the central unit 36, performing the aforementioned conversion and automatic reversal. Two computer programs 40 and 42, which can be selected according to a software switch 44, are shown in FIG. 1.

In accordance with one possible embodiment of the present invention, the first computer program 40 includes instructions for implementing the conversion of the movement instructions provided by the control peripheral 28 into individual instructions for controlling each of the motors M1 to M6, and for implementing the storing of the respective positions of the latter at the desired times. The second computer program 42 includes instructions for implementing the automatic reversal.

It should be noted that the electronic circuit 32 as schematically represented in FIG. 1 can, for example, be implemented in a computer device such as a conventional computer comprising a processor associated with one or more memories for storing data files and computer programs whose instructions are intended to be executed by the processor, such as the instructions of the programs 40, 42 and of the software switch 44, which may also constitute a computer program. These programs are shown as separate, but this distinction is purely functional. They could just as easily be grouped according to any combination into one or more software programs. Their functions could also be at least partly micro-programmed or micro-wired into dedicated integrated circuits. Thus, as an alternative, the computer device implementing the electronic circuit 32 could be replaced by an electronic device made of digital circuits only (without computer programs) for performing the same actions.

More specifically, the first computer program 40 includes instructions 46 for performing a Jacobian conversion of the instructions provided by the control peripheral 28, expressed in the global coordinate system (X0, Y0, Z0), into individual instructions for controlling each of the motors M1 to M6 for actuating the articulated arm 10 using Jacobian parameters stored in memory. This Jacobian converter function is well known to the skilled person and will not be detailed. The individual control instructions provided by execution of the computer program 40 are to be transmitted by the central unit 36 to the articulated arm 10.

The first computer program 40 further includes instructions 48 performing a retrieval, for example at regular time intervals when the surgical instrument 12 is in motion, of the positions of the motors M1 to M6 in order to store them in memory 38, more precisely in a portion 50 of the memory 38 dedicated to data storage. This portion of memory 50 is advantageously structured as a stack, i.e., as a LIFO (Last In First Out) type memory. Data retrieval is carried out by the central unit 36. Thus, the successive positionings of the articulated arm 10 recovered at regular time intervals during the controlled movement of the functional end 24 along the path followed, i.e., more precisely the respective successive positions of the motors M1 to M6 in the example of FIG. 1, are stored in LIFO memory 50 according to an ordered series.

More specifically too, the second computer program 42 includes instructions 52 for performing a readout of the respective successive positions of the motors M1 through M6 stored in LIFO memory 50 for successive step by step reversals of the articulated arm 10 from the latter to the first set of such successive positions. Each time a new set of positions of the motors M1 to M6 is read, allowing the path followed by the functional end 24 of the surgical instrument 12 to be retraced in the opposite direction, the instructions 52 generate the corresponding individual instructions for controlling the motors M1 to M6. These are transmitted to the articulated arm 10 by the central unit 36 and the set of positions read is then deleted from the LIFO memory 50.

The software switch 44 makes it possible to select the execution of one or the other of the computer programs 40, 42 depending on whether the pedal 34 is actuated or not. By default, the first computer program 40 is selected. Any movement instruction provided by the control peripheral 28 is taken into account by the central unit 36 and converted into individual instructions for controlling each of the motors M1 to M6 by execution of this program. In addition, at regular time intervals, the successive positions of the motors M1 to M6 are stored in a stacked ordered list in the LIFO memory 50. By pressing the pedal 34 with the foot, the software switch 44 is set to an execution of the second computer program 42. Any new movement instructions issued by the control peripheral 28 are then no longer taken into account. The successive position data of the motors M1 to M6 stored in LIFO memory 50 are unstacked one after the other in the reverse order of their storing to reconstruct the reverse path followed by the surgical instrument 12. If the electronic device 32 is programmed to be sensitive to the pressure exerted on the pedal 34, the resulting reversal of the robotic arm 10 is, for example, all the faster as the pressure is strong. In a preferred embodiment, as soon as the pedal 34 is released, the automatic reversal stops, whether or not the LIFO memory 50 is cleared, and the software switch 44 switches back to running the first computer program 40.

FIG. 2 illustrates the successive steps of a surgical intervention method using the robotic device of FIG. 1.

At a first step 100, the pedal 34 is not actuated and an operator engages the movement of the functional distal end 24 of the surgical instrument 12 carried by the articulated arm 10 using the control peripheral 28.

At a subsequent step 102, the central processing unit 36 executes the instructions 46 and 48 of the first computer program 40 to convert the instructions provided by the control peripheral 28 into individual instructions for controlling the motors M1 to M6 and to regularly store their successive positions so as to preserve in LIFO memory 50 the path followed by the surgical instrument 12. It should be noted that the fact of recording the successive positions of the motors M1 to M6 at regular time intervals advantageously makes it possible to keep information on the speed of movement of the surgical instrument 12 since the distance covered by its functional distal end 24 between two recordings is proportional to its speed of movement.

At a next step 104, the practitioner wishes to initiate a rapid and automatic reversal of the surgical instrument 12 along the path already completed. To do so, he or she presses the pedal 34.

At a subsequent step 106, the central processing unit 36 then executes the instructions 52 of the second computer program 42 to perform this automatic reversal as previously explained, wherein the speed of the reversal may be a function of the pressure exerted on the pedal 34.

Finally, at a last step 108, the practitioner releases the pedal 34 so that the automatic fast reversal stops, whether or not the LIFO memory 50 is cleared. The method may then resume at step 100 or 104.

FIG. 3 illustrates an example of a scenario realized by executing the method of FIG. 2.

Initially (i.e., at first execution of step 100), the LIFO memory 50 is empty and the functional distal end 24 of the surgical instrument 12 is at an initial point P1.

While performing step 102, the functional distal end 24 moves:
from point P1 to a point P2 at high speed, hence the storing of a small amount of successive movement information in LIFO memory 50; then
from point P2 to a point P3 at average speed, hence the storing of an intermediate number of successive movement information in LIFO memory 50; then
from point P3 to a point P4 at average speed, hence the storing of an intermediate number of successive movement information in LIFO memory 50; then
from point P4 to a point P5 at slow speed, hence the storing of a large amount of successive movement information in LIFO memory 50; then
from point P5 to a point P6 at high speed, hence the recording of a small amount of successive movement information in LIFO memory 50.

At point P6, the operator decides to activate the rapid and automatic reversal of the surgical instrument 12 by pressing the pedal 34 (step 104).

This results in the execution of step 106 during which the functional distal end 24 returns:
from P6 to P5 in fast return, hence the unstacking of the successive movement information previously stored between P5 and P6; then
from P5 to P4 in fast return, hence the unstacking of the successive movement information previously stored between P4 and P5; then
from P4 to P3 in fast return, hence the unstacking of the successive movement information previously stored between P3 and P4.

Back at point P3, the operator decides to interrupt the rapid and automatic reversal of the surgical instrument 12 by releasing the pedal 34 (step 108).

He or she then decides to direct the functional distal end 24 of the surgical instrument 12 to another path and thereby returns to a new execution of step 100.

During a new execution of step 102 marking the end of the scenario illustrated in FIG. 3, the functional distal end 24 moves:
from point P3 to a point P7 at fast speed, resulting in a small amount of successive movement information being stored in LIFO memory 50; and then
from point P7 to a point P8 at a slow speed, resulting in a large amount of successive movement information being stored in LIFO memory 50.

The surgical intervention method in FIG. 2 and its example scenario in FIG. 3 are easily generalizable to the implementation of other interventions than those on the middle or inner ear of a patient.

It clearly appears that a robotic device such as the one described above allows for safe surgical intervention in certain situations where rapid removal of the surgical instrument without wrong movement or deviation from the path previously followed is desired.

It should also be noted that the invention is not limited to the embodiment described above.

It is advantageously applied to the architecture and kinematics of the articulated arm 10 of FIG. 1, but it can be generalized to other architectures and kinematics by adapting the corresponding conversions and positioning information.

The successive positionings of the articulated arm 10 are recorded in the form of motors' positions, but could be recorded in other forms, such as successive 6D positions of the surgical instrument 12.

It will be more generally apparent to those skilled in the art that various modifications can be made to the above-described embodiment in light of the teaching just disclosed. In the above detailed presentation of the invention, the terms used should not be interpreted as limiting the invention to the embodiment set forth in the present description, but should be interpreted to include all equivalents the anticipation of which is within the reach of those skilled in the art by applying their general knowledge to the implementation of the teaching that has just been disclosed to them.

1. A robotic surgical intervention device comprising:
an articulated arm, with actuating motors, a distal end of which is intended to carry a surgical instrument;
a control peripheral of the articulated arm for moving a functional end of the surgical instrument along a path; and
means for processing movement instructions provided by the control peripheral to convert them into individual control instructions for each of the actuating motors of the articulated arm, and
a trigger for automatic reversal of the articulated arm, which can be mechanically actuated by an operator, the actuation of which causes, independently of any movement instruction from the control peripheral, the sending of individual reverse control instructions to each of the actuating motors of the articulated arm for a reverse movement of the functional end of the surgical instrument along the completed path.

2. The robotic surgical intervention device as claimed in claim 1, configured so that actuation of the automatic reversal trigger causes a suspension of processing of new movement instructions provided by the control peripheral

3. The robotic surgical intervention device as claimed in claim 1, wherein:
the processing means include means for storing in memory an ordered series of successive positionings of the articulated arm during controlled movement of the functional end along the path; and
the processing means are configured so that actuation of the automatic reversal trigger causes successive reversals of the articulated arm step by step from a last stored positioning to a first stored positioning of this ordered series.

4. The robotic surgical intervention device as claimed in claim 3, wherein each stored positioning of the ordered series of successive positionings of the articulated arm comprises a set of positions of its actuating motors.

5. The robotic surgical intervention device as claimed in claim 3, wherein the storing means are configured to store in memory the successive positionings of the articulated arm at regular time intervals.

6. The robotic surgical intervention device as claimed in claim 3 wherein the memory in which the ordered series of successive positionings of the articulated arm is stored is configured as a stack structure.

7. The robotic surgical intervention device as claimed in claim 1, wherein the automatic reversal trigger comprises a stop pedal or a push button with a speed variator for varying the speed of the automatic reversal of the articulated arm as a function of pressure exerted by the operator.

8. The robotic surgical intervention device as claimed in claim 7, wherein the processing means are configured to stop the current automatic reversal and resume processing new movement instructions provided by the control peripheral as soon as no pressure is any more applied to the automatic reversal trigger by the operator.

9. The robotic surgical intervention device as claimed in claim 1, wherein the control peripheral is a 6D joystick.

10. The robotic surgical intervention device as claimed in claim 1, configured and dimensioned for a middle or inner ear surgical intervention on a patient, the surgical instrument itself being a patient's middle or inner ear surgical intervention instrument.
